(19) United States
(12) Patent Application Publication
(10) Pub. No.: US 2026/0314653 A1
(43) Pub. Date: Oct. 15, 2026

(54) AN AEROSOL GENERATING MATERIAL

(71) Applicant: NICOVENTURES TRADING LIMITED, London (GB)

(72) Inventors: Peter DAVIS, London (GB); Agustin PIAGGIO, London (GB); Tom STACEY, London (GB); Erick REIS, London (GB); Jennifer Louise SCIROCCO, London (GB)

(21) Appl. No.: 19/149,346

(22) PCT Filed: Jan. 30, 2024
(86) PCT No.: PCT/GB2024/050242

§ 371 Date: Jul. 18, 2025

(30) Foreign Application Priority Data
Jan. 31, 2023 (GB) 2301400.4

(51) Int. Cl. A24B 15/30; A24B 15/16; A24B 15/32; A24D 1/20; A61K 31/00

(52) U.S. Cl. CPC A24B 15/303; A24B 15/16; A24B 15/32; A24D 1/20; A61K 31/658

(57) ABSTRACT

Described herein is an aerosol generating material comprising: a fibrous material and a non-nicotine active, and a rod, an article, and a delivery system comprising the same.

### TECHNICAL FIELD

The present disclosure relates to an aerosol generating material comprising a fibrous material and non-combustible aerosol-provision systems comprising the aerosol generating material.

### BACKGROUND

Aerosol generating materials are typically heated, for example by a non-combustible aerosol-provision system, to form an aerosol, which may be inhaled by a consumer.

Aerosol generating materials may be made from various sources, including from tobacco material and/or a non-tobacco material.

### SUMMARY

According to a first aspect, there is described an aerosol generating material comprising a fibrous material and a non-nicotine active.

According to a second aspect, there is described an aerosol generating rod comprising an aerosol generating material according to the first aspect.

According to a third aspect, there is described an article comprising an aerosol generating material according to the first aspect or an aerosol generating rod according to the second aspect.

According to a fourth aspect, there is described a delivery system comprising an aerosol generating material according to the first aspect, an aerosol generating rod according to the second aspect, or an article according to the third aspect.

### DESCRIPTION OF DRAWINGS

Embodiments of the invention are described, by way of example only, with reference to the accompanying drawings, in which:

FIG. 1 shows a flowchart for a method of making an aerosol generating material.

### DETAILED DESCRIPTION

The invention relates to aerosol generating materials. The invention also relates to a method of manufacturing an aerosol generating material, the use of an aerosol generating material, an article for use in a delivery system, and a system comprising an aerosol generating material and a device.

Aerosol generating materials comprise aerosolisable components. In use, the aerosol generating material produces an aerosol, for example a suspension of liquid droplets or particulates in a gas. The aerosol comprises a non-nicotine active as well as other components generated by the aerosol generating material. In use, the user inhales the aerosol. As such, it is important that the aerosol generating material produces an aerosol that delivers an appropriate user experience and satisfaction.

The composition of the aerosol contributes to the user's experience and satisfaction. One attribute that contributes to the user's experience and satisfaction is the type of active in the aerosol, for example what physiological or psychological the active may impart on a user. Another attribute that contributes to the user's experience and satisfaction is the perceived harshness of the aerosol.

Certain actives, when aerosolised, may result in an aerosol which has a perceived harshness which is too high for a consumer. Alternatively, certain actives may provide little, to no, perceived harshness, which may be preferred, or disliked, by consumers. For example, it may be preferred to control the content and harshness of an active present in an aerosol.

In an embodiment, an aerosol generating material comprises a fibrous material and a non-nicotine active. Without wishing to be bound by theory, it is hypothesised that inclusion of a fibrous material with a non-nicotine active can impact the perceived harshness of an aerosol, and selecting appropriate fibrous materials may support the generation of a favourable aerosol.

In some instances, it has been found that an aerosol generating material comprising a non-nicotine active may produce an aerosol which has a perceived harshness which is too high for consumers. For example, actives such as cannabinoids or terpenes, may be perceived as being too harsh when aerosolised and inhaled by a consumer. In other embodiments, non-nicotine actives, such as caffeine, taurine, vitamins, and other actives classed as nutraceuticals, may produce an aerosol which is perceived as too harsh by a consumer.

As used herein, the term “delivery system” is intended to encompass systems that deliver a substance to a user, and includes:
combustible aerosol provision systems, such as cigarettes, cigarillos, cigars, and tobacco for pipes or for roll-your-own or for make-your-own cigarettes (whether based on tobacco, tobacco derivatives, expanded tobacco, reconstituted tobacco, tobacco substitutes or other smokable material);
non-combustible aerosol provision systems that release compounds from an aerosol-generating material without combusting the aerosol-generating material, such as electronic cigarettes, tobacco heating products, and hybrid systems to generate aerosol using a combination of aerosol-generating materials; and
aerosol-free delivery systems, such as lozenges, gums, patches, articles comprising inhalable powders, and smokeless tobacco products such as snus and snuff, which deliver a material to a user without forming an aerosol.

According to the present disclosure, a “combustible” aerosol provision system is one where a constituent aerosol-generating material of the aerosol provision system (or component thereof) is combusted or burned during use in order to facilitate delivery of at least one substance to a user.

In some embodiments, the delivery system is a combustible aerosol provision system, such as a system selected from the group consisting of a cigarette, a cigarillo and a cigar.

In some embodiments, the disclosure relates to a component for use in a combustible aerosol provision system, such as a filter, a filter rod, a filter segment, a tobacco rod, a spill, an aerosol-modifying agent release component such as a capsule, a thread, or a bead, or a paper such as a plug wrap, a tipping paper or a cigarette paper.

According to the present disclosure, a “non-combustible” aerosol provision system is one where a constituent aerosol-generating material of the aerosol provision system (or component thereof) is not combusted or burned in order to facilitate delivery to a user.

In some embodiments, the delivery system is a non-combustible aerosol provision system, such as a powered non-combustible aerosol provision system.

In some embodiments, the non-combustible aerosol provision system is an electronic cigarette, also known as a vaping device or electronic nicotine delivery system (END).

It is understood that such an electronic cigarette, or vaping device, need not be restricted for use with nicotine, but can be utilised to aerosolise non-nicotine actives.

In some embodiments, the non-combustible aerosol provision system is a tobacco heating system, also known as a heat-not-burn system. Again, it would be understood that such a system can be used to aerosolise non-nicotine actives.

In some embodiments, the non-combustible aerosol provision system is a hybrid system to generate aerosol using a combination of aerosol-generating materials, one or a plurality of which may be heated. Each of the aerosol-generating materials may be, for example, in the form of a solid, liquid or gel. In some embodiments, the hybrid system comprises a liquid or gel aerosol-generating material and a solid aerosol-generating material. The solid aerosol-generating material may comprise, for example, tobacco material, a non-tobacco product, or a non-nicotine active.

Typically, the non-combustible aerosol provision system may comprise a non-combustible aerosol provision device, also referred to herein as an aerosol generation device, and a consumable for use with the non-combustible aerosol provision system.

In some embodiments, the disclosure relates to consumables comprising aerosol-generating material and configured to be used with non-combustible aerosol provision devices. These consumables are sometimes referred to as articles throughout the disclosure.

In some embodiments, it is envisaged that consumables which themselves comprise a means for powering an aerosol generating component may themselves form the non-combustible aerosol provision system.

In some embodiments, the non-combustible aerosol provision system may comprise a power source and a controller. The power source may be an electric power source or an exothermic power source. In some embodiments, the exothermic power source comprises a carbon substrate which may be energised so as to distribute power in the form of heat to an aerosol-generating material or heat transfer material in proximity to the exothermic power source. In some embodiments, the power source, such as an exothermic power source, is provided in the article so as to form the non-combustible aerosol provision system.

In some embodiments, the non-combustible aerosol provision system may comprise an area for receiving the consumable, an aerosol generator, an aerosol generation area, a housing, a mouthpiece, a filter and/or an aerosol-modifying agent.

In some embodiments, the consumable for use with the non-combustible aerosol provision device may comprise aerosol-generating material, an aerosol-generating material storage area, an aerosol-generating material transfer component, an aerosol generator, an aerosol generation area, a housing, a wrapper, a filter, a mouthpiece, and/or an aerosol-modifying agent.

The substance to be delivered comprises a non-nicotine active substance.

The active substance as used herein may be a physiologically active material, which is a material intended to achieve or enhance a physiological response. The active substance may for example be selected from nutraceuticals, nootropics, psychoactives. The active substance may be naturally occurring or synthetically obtained. The active substance may comprise for example caffeine, taurine, theine, vitamins such as B6 or B12 or C, melatonin, cannabinoids, or constituents, derivatives, or combinations thereof. The active substance may comprise one or more constituents, derivatives or extracts of cannabis or another botanical.

In some embodiments the active substance is a legally permissible recreational drug.

In some embodiments, the non-nicotine active comprises caffeine, taurine, L-theanine, vitamin B1, vitamin B2, vitamin B5, vitamin B6, vitamin B7, vitamin C, ginseng extract, chamomile extract, lemon balm extract, and mixtures thereof.

As noted herein, the active substance may comprise one or more constituents, derivatives or extracts of cannabis, such as one or more cannabinoids or terpenes.

In some embodiments, the non-nicotine active comprises a cannabinoid selected from the list consisting of cannabigerol (CBG), cannabichromene (CBC), cannabidiol (CBD), tetrahydrocannabinol (THC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethylether (CBGM), cannabinerolic acid, cannabidiolic acid (CBDA), cannabinol propyl variant (CBNV), cannbitriol (CBO), tetrahydrocannabinolic acid (THCA), and tetrahydrocannabivarinic acid (THCV A), and mixtures thereof.

The active substance may be cannabidiol, i.e. CBD, or a derivative thereof.

The active substance may be tetrahydrocannabinol, i.e. THC, or a derivative thereof.

As used herein, the term “botanical” includes any material derived from plants including, but not limited to, extracts, leaves, bark, fibres, stems, roots, seeds, flowers, fruits, pollen, husk, shells or the like. Alternatively, the material may comprise an active compound naturally existing in a botanical, obtained synthetically. The material may be in the form of liquid, gas, solid, powder, dust, crushed particles, granules, pellets, shreds, strips, sheets, or the like. Example botanicals are tobacco, eucalyptus, star anise, hemp, cocoa, cannabis, fennel, lemongrass, peppermint, spearmint, rooibos, chamomile, flax, ginger, Ginkgo biloba, hazel, hibiscus, laurel, licorice (liquorice), matcha, mate, orange skin, papaya, rose, sage, tea such as green tea or black tea, thyme, clove, cinnamon, coffee, aniseed (anise), basil, bay leaves, cardamom, coriander, cumin, nutmeg, oregano, paprika, rosemary, saffron, lavender, lemon peel, mint, juniper, elderflower, vanilla, wintergreen, beefsteak plant, curcuma, turmeric, sandalwood, cilantro, bergamot, orange blossom, myrtle, cassis, valerian, pimento, mace, damien, marjoram, olive, lemon balm, lemon basil, chive, carvi, verbena, tarragon, geranium, mulberry, ginseng, theanine, theacrine, maca, ashwagandha, damiana, guarana, chlorophyll, baobab or any combination thereof. The mint may be chosen from the following mint varieties: Mentha Arventis, Mentha c.v., Mentha niliaca, Mentha piperita, Mentha piperita citrata c.v., Mentha piperita c.v, Mentha spicata crispa, Mentha cardifolia, Memtha longifolia, Mentha suaveolens variegata, Mentha pulegium, Mentha spicata c.v. and Mentha suaveolens

In some embodiments, the active substance comprises or is derived from one or more botanicals or constituents, derivatives or extracts thereof and the botanical is selected from eucalyptus, cocoa and hemp.

In some embodiments, the active substance comprises or is derived from one or more botanicals or constituents, derivatives or extracts thereof and the botanical is selected from star anise, rooibos, mint, and fennel.

In an embodiment, an aerosol generating material comprises: a fibrous material and a non-nicotine active.

In some embodiments, the aerosol generating material comprises a total fibrous material content of between about 70% and about 95% by weight on a dry weight basis.

An aerosol is a suspension of particles of liquid, solid, or both, within a gas.

In some embodiments, the substance to be delivered comprises a flavour.

In some embodiments, the non-nicotine active is a flavour.

As used herein, the terms “flavour” and “flavourant” refer to materials which, where local regulations permit, may be used to create a desired taste, aroma or other somatosensorial sensation in a product for adult consumers. They may include naturally occurring flavour materials, botanicals, extracts of botanicals, synthetically obtained materials, or combinations thereof (e.g., tobacco, cannabis, licorice (liquorice), hydrangea, eugenol, Japanese white bark magnolia leaf, chamomile, fenugreek, clove, maple, matcha, menthol, Japanese mint, aniseed (anise), cinnamon, turmeric, Indian spices, Asian spices, herb, wintergreen, cherry, berry, red berry, cranberry, peach, apple, orange, mango, clementine, lemon, lime, tropical fruit, papaya, rhubarb, grape, durian, dragon fruit, cucumber, blueberry, mulberry, citrus fruits, Drambuie, bourbon, scotch, whiskey, gin, tequila, rum, spearmint, peppermint, lavender, aloe vera, cardamom, celery, cascarilla, nutmeg, sandalwood, bergamot, geranium, khat, naswar, betel, shisha, pine, honey essence, rose oil, vanilla, lemon oil, orange oil, orange blossom, cherry blossom, cassia, caraway, cognac, jasmine, ylang-ylang, sage, fennel, wasabi, piment, ginger, coriander, coffee, hemp, a mint oil from any species of the genus Mentha, eucalyptus, star anise, cocoa, lemongrass, rooibos, flax, Ginkgo biloba, hazel, hibiscus, laurel, mate, orange skin, rose, tea such as green tea or black tea, thyme, juniper, elderflower, basil, bay leaves, cumin, oregano, paprika, rosemary, saffron, lemon peel, mint, beefsteak plant, curcuma, cilantro, myrtle, cassis, valerian, pimento, mace, damien, marjoram, olive, lemon balrn, lemon basil, chive, carvi, verbena, tarragon, limonene, thymol, camphene), flavour enhancers, bitterness receptor site blockers, sensorial receptor site activators or stimulators, sugars and/or sugar substitutes (e.g., sucralose, acesulfame potassium, aspartame, saccharine, cyclamates, lactose, sucrose, glucose, fructose, sorbitol, or mannitol), and other additives such as charcoal, chlorophyll, minerals, botanicals, or breath freshening agents. They may be imitation, synthetic or natural ingredients or blends thereof. They may be in any suitable form, for example, liquid such as an oil, solid such as a powder, or gas.

In some embodiments, the flavour comprises menthol, spearmint and/or peppermint. In some embodiments, the flavour comprises flavour components of cucumber, blueberry, citrus fruits and/or redberry. In some embodiments, the flavour comprises eugenol. In some embodiments, the flavour comprises flavour components extracted from tobacco. In some embodiments, the flavour comprises flavour components extracted from cannabis, such as terpenes.

In some embodiments, the flavour may comprise a sensate, which is intended to achieve a somatosensorial sensation which are usually chemically induced and perceived by the stimulation of the fifth cranial nerve (trigeminal nerve), in addition to or in place of aroma or taste nerves, and these may include agents providing heating, cooling, tingling, numbing effect. A suitable heat effect agent may be, but is not limited to, vanillyl ethyl ether and a suitable cooling agent may be, but not limited to eucolyptol, WS-3.

In some embodiments, the flavour comprises menthol, spearmint and/or peppermint. In some embodiments, the flavour comprises flavour components of berry fruits, citrus fruits, and/or tropical fruits. In some embodiments, the flavour may comprise cucumber, blueberry, citrus fruits, pineapple, strawberry, and/or redberry. In an embodiment, the aerosol generating material comprises menthol.

In some embodiments, the flavour added to an aerosol generating material may be selected to enhance the underlying properties of the aerosol generating material. For example, as described herein, it may be preferable that the fibrous material comprises a botanical material. In such instances, the botanical material may produce a particularly neutral aroma profile, for example when rooibos, fennel, star anise, and/or mint are utilised in the aerosol generating material. When one, or more, of these botanicals is used, the aroma may be enhanced when paired with a flavour such as menthol, spearmint and/or peppermint; berry fruits, citrus fruits, and/or tropical fruits, or any combination of these flavours.

An aerosol generating material is a material that is capable of generating aerosol, for example when heated, irradiated or energized in any other way. Aerosol generating materials may, for example, be in the form of a solid, liquid or semi-solid (such as a gel) which may or may not contain an active substance and/or flavourants.

The aerosol-generating material may comprise one or more active substances and/or flavours, one or more aerosol-former materials, and optionally one or more other functional material.

The aerosol generating material may comprise, or be, a continuous sheet of material. The sheet may be in the form of a wrapper, it may be gathered to form a gathered sheet or it may be shredded to form a shredded sheet. The shredded sheet may comprise one or more strands or strips of aerosol generating material.

The sheet or shredded sheet comprises a first surface and a second surface opposite the first surface. The dimensions of the first and second surfaces are congruent. The first and second surfaces of the sheet or shredded sheet may have any shape. For example, the first and second surfaces may be square, rectangular, oblong or circular. Irregular shapes are also envisaged.

The first and/or second surfaces of the sheet or shredded sheet may be relatively uniform (e.g. they may be relatively smooth) or they may be uneven or irregular. For example, the first and/or second surfaces of the sheet may be textured or patterned to define a relatively coarse surface. In some embodiments, the first and/or second surfaces are relatively rough.

The smoothness of the first and second surfaces may be influenced by a number of factors, such as the area density of the sheet or shredded sheet, the nature of the components that make up the aerosolisable material or whether the surfaces of the material have been manipulated, for example embossed, scored or otherwise altered to confer them with a pattern or texture.

The areas of the first and second surfaces are each defined by a first dimension (e.g. a width) and a second dimension (e.g. a length). The measurements of the first and second dimensions may have a ratio of 1:1 or greater than 1:1 and thus the sheet or shredded sheet may have an “aspect ratio” of 1:1 or greater than 1:1. As used herein, the term “aspect ratio” is the ratio of a measurement of a first dimension of the first or second surface to a measurement of a second dimension of the first or second surface. An “aspect ratio of 1:1” means that a measurement of the first dimension (e.g. width) and a measurement of the second dimension (e.g. length) are identical. An “aspect ratio of greater than 1:1” a measurement of the first dimension (e.g. width) and a measurement of the second dimension (e.g. length) are different. In some embodiments, the first and second surfaces of the sheet or shredded sheet have an aspect ratio of greater than 1:1, such as 1:2, 1:3, 1:4, 1:5, 1:6, 1:7 or more.

The shredded sheet may comprise one or more strands or strips of the aerosolisable material. In some embodiments, the shredded sheet comprises a plurality (e.g. two or more) strands or strips of the aerosolisable material. The strands or strips of aerosolisable material may have an aspect ratio of 1:1. In an embodiment, the strands or strips of aerosolisable material have an aspect ratio of greater than 1:1. In some embodiments, the strands or strips of aerosolisable material have an aspect ratio of from about 1:5 to about 1:16, or about 1:5, 1:6, 1:7, 1:8, 1:9, 1:10, 1:11 or 1:12. Where the aspect ratio of the strands or strips is greater than 1:1, the strands or strips comprises a longitudinal dimension, or length, extending between a first end of the strand or strip and a second end of the strand or strip.

Where the shredded sheet comprises a plurality of strands or strips of material, the dimensions of each strand or strip may vary between different strands or strips. For example, the shredded sheet may comprise a first population of strands or strips and a second population of strands or strips, wherein the dimensions of the strands or strips of the first population are different to the dimensions of the strands or strips of the second population. In other words, the plurality of strands or strips may comprise a first population of strands or strips having a first aspect ratio and a second population of strands or strips having a second aspect ratio that is different to the first aspect ratio.

A first dimension, or cut width, of the strands or strips of aerosolisable material is between 0.9 mm and 1.5 mm. The inventors have found that, when strands or strips of aerosolisable material having a cut width of below 0.9 mm are incorporated into an article for use in a non-combustible aerosol provision system, the pressure drop across the article may be increased to a level that renders the article unsuitable for use in a non-combustible aerosol-provision device. However, if the strands or strips have a cut width above 2 mm (e.g. greater than 2 mm), then it may be challenging to insert the strands or strips of aerosolisable material into the article during its manufacture. In a preferred embodiment, the cut width of the strands or strips of aerosolisable material is between about 1 mm and 1.5 mm.

The strands or strips of material are formed by shredding the sheet of aerosolisable material. The sheet of aerosolisable material may be cut width-wise, for example in a cross-cut type shredding process, to define a cut length for the strands or strips of aerosolisable material, in addition to a cut width. The cut length of the shredded aerosolisable material is preferably at least 5 mm, for instance at least 10 mm, or at least 20 mm. The cut length of the shredded aerosolisable material can be less than 60 mm, less than 50 mm, or less than 40 mm.

In some embodiments, a plurality of strands or strips of aerosolisable material is provided and at least one of the plurality of strands or strips of aerosolisable material has a length greater than about 10 mm. At least one of the plurality of strands or strips of aerosolisable material can alternatively or in addition have a length between about 10 mm and about 60 mm, or between about 20 mm and about 50 mm. Each of the plurality of strands or strips of aerosolisable material can have a length between about 10 mm and about 60 mm, or between about 20 mm and about 50 mm.

A consumable is an article comprising or consisting of aerosol-generating material, part or all of which is intended to be consumed during use by a user. In some embodiments, the consumable for use with a non-combustible aerosol provision device may comprise one or more other components, such as, an aerosol-generating material storage area, an aerosol-generating material transfer component, an aerosol generator, an aerosol generation area, a housing, a wrapper, a filter, a mouthpiece, and/or an aerosol-modifying agent. A consumable may also comprise an aerosol generator, such as a heater, that emits heat to cause the aerosol-generating material to generate aerosol in use. The heater may, for example, comprise combustible material, a material heatable by electrical conduction, or a susceptor.

A susceptor is a material that is heatable by penetration with a varying magnetic field, such as an alternating magnetic field. The susceptor may be an electrically-conductive material, so that penetration thereof with a varying magnetic field causes induction heating of the heating material. The heating material may be magnetic material, so that penetration thereof—with a varying magnetic field causes magnetic hysteresis heating of the heating material. The susceptor may be both electrically-conductive and magnetic, so that the susceptor is heatable by both heating mechanisms. The device that is configured to generate the varying magnetic field is referred to as a magnetic field generator, herein.

An aerosol generator is a heater capable of interacting with the aerosol-generating material so as to release one or more volatiles from the aerosol-generating material to form an aerosol. In some embodiments, the aerosol generator is capable of generating an aerosol from the aerosol-generating material without heating. For example, the aerosol generator may be capable of generating an aerosol from the aerosol-generating material without applying heat thereto, for example via one or more of vibrational, mechanical, pressurisation or electrostatic means.

A heater may be provided in a portion of the housing of delivery system. The heater may comprise a base portion which may be located in a recess provided in a portion of the body of the device and the heater may extend, or project, into the heating chamber. The heater may comprise an elongate heater in the form of a pin. Alternatively, the heater may comprise other elongate configurations, such as a blade.

In some embodiments, the heating arrangement may be a resistive heating arrangement. In such an arrangement, the heating assembly comprises a resistive heating generator including components to heat the heater element via a resistive heater element. In this case, an electrical current is directly applied to a resistive heater element, and the resulting flow of current in the heater element, acting as a heating component, causes the heater element to be heated by Joule heating. The resistive heater element comprises resistive material configured to generate heat when a suitable electrical current passes through it, and the heating arrangement comprises electrical contacts for supplying electrical current to the resistive material. In an embodiment, the resistive heater element transfers heat to the heater, for example by conduction. The provision of a resistive heating arrangement allows for a compact arrangement. Resistive heating provides an efficient configuration.

In some embodiments, the heating arrangement may be inductive heating. Induction heating is a process of heating an electrically conducting heating member, such as a susceptor, by electromagnetic induction. An induction heating arrangement may comprise an inductive element, for example, one of more inductor coils, and a device for passing a varying electric current, such as an alternating electric current, through the inductive element. The varying electric current in the inductive element produces a varying magnetic field. The varying magnetic field penetrates a susceptor suitably positioned with respect to the inductive element. In inductive heating, as compared to heating by conduction for example, heat is generated inside the susceptor, allowing for rapid heating. Furthermore, there need not be any physical contact between the inductive element and the susceptor, allowing for enhanced freedom in construction and application.

In some embodiments, the fibrous material comprises a botanical material. A fibrous material comprising a botanical material may supplement the flavour profile of any aerosol produced. For example, a botanical material may be selected which has a relatively neutral flavour profile. A neutral flavour profile may be described as a flavour profile which contains few intense flavours and/or one which readily accepts the loading of top flavours, without significantly affecting their perception. For example, a fibrous material containing reconstituted tobacco material may be described as producing an aerosol with a relatively neutral flavour profile. It may be advantageous to be able to produce an aerosol which has a similarly neutral flavour profile to that of reconstituted tobacco, but without using tobacco material.

In some embodiments, the fibrous material may be called a substrate. It may be preferable for the substrate to be made from a non-tobacco material, such that a consumer may reduce their use of tobacco-based materials. Consumers may also wish to reduce their use of nicotine, and therefore seek to use a non-nicotine active. Certain consumers may desire certain non-nicotine actives, such as cannabinoids, due to the physiological and potentially psychological effects that such actives may deliver.

In some embodiments, the botanical material may be any non-tobacco botanical material. For example, the material may be derived from species which are members of the Asteracae family, the Fabaceae family, the Myrtaceae family, Apiaceae family, Camellia taliensis, the Solanaceae family, the Brassicaceae family, the Caricaceae family, the Asclepiadaceae family, the Equisetaceae family, the Oleaceae family, the Lamiaceae family, and tisanes. For example, the non-tobacco botanical material may be selected from the Matricaria species, such as chamomile; the Pimpinella anisum species, such as anise; the Foeniculum vulgare species, such as fennel; jasmine; lavender; cloves; eucalyptus, and the species Aspalathus linearis, such as rooibos.

In some embodiments, the fibrous material comprises rooibos.

In some embodiments, the non-tobacco botanical material is selected from a botanical material which comprises favourable aroma properties for use in a non-combustible aerosol provision system. For example, the non-tobacco botanical material may comprise relatively few aroma compounds compared to traditional tobacco material; therefore, an aerosol produced from a non-tobacco botanical material may have a different profile of volatile compounds compared to an aerosol produced from a tobacco material. The non-tobacco botanical material may deliver an aerosol which is considered favourable by a consumer of tobacco-based delivery systems. In some embodiments, a non-tobacco botanical material may produce an aerosol, when heated, with a sensorial experience that is comparable to that provided by a conventional combustible product, such as a cigarette. In some embodiments, the non-tobacco botanical material is selected from seed-producing plants which do not develop persistent woody tissue and which are often valued for their medicinal or sensorial characteristics. In some embodiments, it may be preferable to provide an aerosol generating material, for use in a non-combustible aerosol provision system which does not comprise any tobacco plant material.

As used herein, the term “tobacco material” refers to any material comprising tobacco or derivatives or substitutes thereof. The tobacco material may be in any suitable form.

The term “tobacco material” may include one or more of tobacco, tobacco derivatives, expanded tobacco, reconstituted tobacco or tobacco substitutes. The tobacco material may comprise one or more of ground tobacco, tobacco fibre, cut tobacco, extruded tobacco, tobacco stem, tobacco lamina, and/or reconstituted tobacco. It is understood that a nicotine extract, derived from tobacco material, is not considered to be a “tobacco material”.

In some embodiments, it may be preferred that the fibrous material does not contain tobacco material; certain consumers may prefer to inhale an aerosol which has been produced from a non-tobacco material.

The present inventors have identified that the perceived harshness of an aerosol can be modified based on the pH of the aerosol generating material. For example, it may be preferable for an aerosol generating material to exhibit a pH which is acidic, neutral, or alkaline, depending on the non-nicotine active present and the stability of the active.

In some embodiments, upon contact with purified water, the aerosol generating material may give a pH of at most about 6.5, such as a pH of at most about 6.0, such as a pH of at most about 5-5, such as a pH of at most about 5.

In some embodiments, upon contact with purified water, the aerosol generating material may give a pH of between about 5 and about 8; such as a pH of between about 5-5 and about 7.5; such as a pH of between about 6.0 and about 7.0.

In some embodiments, upon contact with purified water, the aerosol generating material may give a pH of at least about 6.5; such as a pH of at least about 7.0; such as a pH of at least about 7.5; such as a pH of at least about 8.0; such as a pH of at least about 8.5.

In particular, it has been identified that certain non-nicotine actives, such as cannabinoids, may be more stable in their natural acidic form. For example, inclusion of cannabidiolic acid (CBDA) may be preferred over cannabidiol because it is naturally more stable. Use of CBDA may inherently reduce the pH of the aerosol generating material. In some instances, use of a non-acidic form of an active may be preferred.

It is understood that measuring of the pH of a solid material is not possible and that the material must be introduced into a solvent, where the pH of the solvent may be measured, in order to determine the pH of the original material. The pH of an aerosol generating material may be measured by dissolving or dispersing the aerosol generating material in purified water and measuring the pH using a conventional pH meter. The pH may be determined by adding 100 g of aerosol generating material to purified water, such as 500 mL of purified water. This sample is shaken, or stirred, for 30 mins, after which the pI is measured using a conventional pH meter. The pH is measured at room temperature and pressure.

In some embodiments, the aerosol generating material comprises a total fibrous material content of between about 700% and about 95% by weight on a dry basis. For example, the total fibrous material content may be between about 70% and about 90%, such as between about 70% and about 85%, such as between about 75% and about 80% by weight on a dry basis. In some embodiments, the total fibrous material content of the aerosol generating material is about 75%, by weight on a dry basis.

In some embodiments, the fibrous material comprises first and second fibrous materials. In some embodiments, the first and second fibrous materials are different. When the first and second fibrous materials are different, the properties of the aerosol generating material, e.g. the aerosol generated, may be modified. For example, two different fibrous materials could be combined which complement each other and provide an aerosol that is particularly pleasant/favourable to a consumer.

In some embodiments, the first fibrous material comprises a non-tobacco botanical material and the second fibrous material comprises wood fibres/wood pulp. For example, the non-tobacco botanical material may be selected from any botanical material discussed herein; preferably, the non-tobacco botanical material is selected from the list consisting of fennel, star anise, mint, and rooibos, and mixtures thereof.

Used herein, wood fibre and wood pulp may be used to describe a cellulose material derived from a cellulose material which has little, or substantially no, noticeable aroma. For example, wood fibre and wood pulp may be of similar nature to wood fibre/wood pulp used to make paper. Wood fibre and wood pulp are obtained from a non-tobacco material.

In some embodiments, a ratio between the first fibrous material and the second fibrous material may be from about 10:1 to about 2:1. For example, the ratio may be from about 9:1 to about 3:1; such as from about 8:1 to about 4:1; such as from about 7:1 to about 4:1; such as from about 6:1 to about 4:1. In some embodiments, a ratio between the first fibrous material and the second fibrous material may be about 5:1.

In some embodiments, modifying the amount of a first fibrous material, comprising, for example, a botanical material; and a second fibrous material, comprising, for example, wood fibres/wood pulp, enables the aerosol to be modified/tailored to a specific desirable profile. For example, one may increase the relative amount of a non-tobacco botanical material, compared to wood fibre/wood pulp, therefore increasing the amount of aroma compounds derived from the non-tobacco botanical material in an aerosol.

The correct amount of an organic acid to be used in an aerosol-generating material can be defined in a number of ways. For example, the amount of organic acid used may be defined in relation to the aerosol generating material, for example as a weight percentage of the aerosol generating material. Alternatively, the amount of organic acid may be defined in relation to the non-nicotine active content of the aerosol generating material, for example by reference to a ratio of the moles of non-nicotine active to the moles of acid.

Without wishing to be bound by any particular theory, it is understood that when the amount of acid in the aerosol-generating material is as described herein, the aerosol produced by the aerosol-generating material contains a ratio of non-nicotine active in the gas phase to non-nicotine active in the particulate/liquid phase (non-nicotine active gas):non-nicotine active (particulate/liquid)) that is particularly beneficial. That is, the ratio of non-nicotine active (gas):non-nicotine active (particulate/liquid) in the aerosol produced by the present aerosol generating materials provides an improved user experience and satisfaction. This is believed to be because using an amount of acid as defined herein allows for optimal protonation of the non-nicotine active. The aerosols produced by the aerosol-generating materials described herein deliver an appropriate amount of non-nicotine active to the user. Furthermore, users report that such aerosol-generating materials are neither too harsh, nor not harsh enough. As such, the aerosol-generating materials comprising an acid as described herein produce an aerosol with an appropriate perceived harshness.

In some embodiments, the acid is selected from the group consisting of levulinic acid, lactic acid, benzoic acid, citric acid, 2-methylbutyric acid, or 2-methylvaleric acid. In some embodiments, the acid is benzoic acid. In some embodiments, the acid is levulinic acid.

The term lactic acid is synonymous with the term 2-hydroxypropanoic acid and covers both D and L enantiomers separately or a mixture thereof. For example, the lactic acid can be a mixture (for example a racemic mixture) of D-2-hydroxypropanoic acid and L-2-hydroxypropanoic acid. The term levulinic acid is synonymous with the term 4-oxopentanoic acid.

In particular, it is understood that the total amount of acid as defined herein represents the lowest amount of acid required to provide the appropriate sensory experience.

Using an amount of acid lower than that described herein results in a non-optimal amount of active reaching the user.

The aerosol generating material comprises an organic acid. The total amount of the acid is from about 0.1% to about 5% by weight of the aerosol-generating material. For example, the total amount of the acid is from about 0.1% to about 5%, from about 0.5% to about 5%, from about 1% to about 5%, from about 1.5% to about 5%, from about 2% to about 5%, or from about 2.5% to about 5% by weight of the aerosol-generating material. For example, the total amount of the acid is from about 2.5% to about 5%, from about 2.5% to about 4.5%, from about 2.5% to about 4%, from about 2.5% to about 3.5%, or from about 2.5% to about 3% by weight of the aerosol-generating material.

In some embodiments, the aerosol generating material comprises the acid in an amount (i.e. moles of acid) from about 50%, about 60%, about 70%, about 80%, about 90%, about 100%, about 110%, about 120%, about 130%, about 140%, about 150%, about 160%, about 170%, about 180%, or about 190% to about 200% relative to the moles of non-nicotine active.

In some embodiments, the aerosol generating material comprises an extract derived from a non-tobacco botanical material.

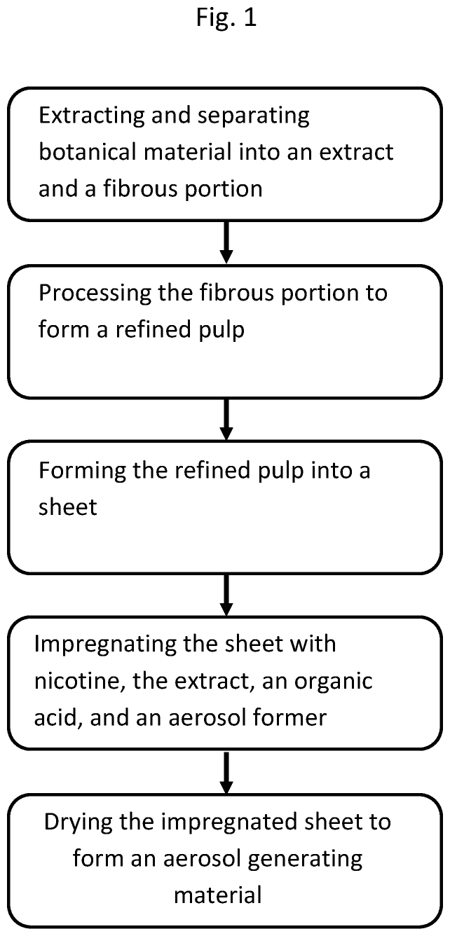

In some embodiments, when the aerosol generating material comprises a botanical material, the aerosol generating material further comprises an extract derived from the botanical material. For example, as shown in FIG. 1, the aerosol generating material may be prepared by extracting and separating the botanical material into an extract and a fibrous portion. The fibrous portion may be combined with a second fibrous material, processed to form a refined pulp, and formed into a sheet. The extract, in addition to a non-nicotine active, an organic acid, and an aerosol former may be applied to, e.g. impregnated into, the sheet which is subsequently dried. Reintroducing the extract from the botanical material into the resulting aerosol generating material enables the aroma compounds from the botanical material to be present in the aerosol generating material. It is thought that processing of a botanical material, to form an aerosol generating material, for example by a typical paper-making process, results in the loss of volatile flavour/aroma compounds. In particular, heating of the botanical material during a paper-making process can result in the loss of highly volatile compounds, which are more likely to be the compounds which provide the flavour and/or aroma to the botanical material.

It is understood that the process for manufacturing an aerosol generating material can be modified depending on the active included. For example, certain non-nicotine actives such as cannabinoids like CBD and THC are lipophilic and therefore may not be easily incorporated in an aerosol generating material according to the paper-making process set out above. Such actives can be incorporated in a process including the steps of extracting and separating the botanical material into an extract and a fibrous portion; processing the fibrous portion to form a refined pulp, and forming the refined pulp into a sheet; adding the extract and an aerosol former to the sheet to form a impregnated sheet; drying the sheet; and adding the non-nicotine active, for example by mixing the active with the sheet, spraying the active onto the sheet, or any other way of distributing the active onto/into the sheet.

Furthermore, it is understood that certain actives may be particularly sensitive to elevated temperatures. Therefore, the drying step of the process set out in FIG. 1 can be adjusted, in order to ensure that any non-nicotine active remains within the sheet and is not lost. In some embodiments, the heating step may be performed at a temperature below about 120° C., such as below about 110° C., such as below about 100° C., such as below about 90° C., such as below about 80° C., such as below about 70° C., such as below about 60° C., such as below bout 50° C., such as below about 40° C. In some embodiments, the aerosol generating material contains essentially 100% of a non-tobacco botanical material. For example, the fibrous material comprises essentially all of the fibrous portion of the original botanical material and the extract comprises essentially the entire extract removed from the original botanical material. Utilising all of the fibrous portion and the extract of a botanical material is favourable as waste material is reduced. Furthermore, the full flavour profile of the original botanical material is maintained in the resulting aerosol generating material. This may be particularly favoured by consumers who may desire to consume an aerosol which has an aroma from a botanical material.

In some embodiments, the aerosol generating material comprises an extract in an amount of from about 5% to about 15% by weight of the aerosol generating material. For example, the extract may be included in an amount of from about 6% to about 14% by weight of the aerosol generating material; such as from about 7% to about 13% by weight of the aerosol generating material; such as from about 8% to about 12% by weight of the aerosol generating material; such as from about 10% to about 12% by weight of the aerosol generating material. In some embodiments, the aerosol generating material comprises an extract in an amount of about 11% by weight of the aerosol generating material.

The aerosol generating material comprises an aerosol former/aerosol former material. The total amount of aerosol former may be from about 10% to about 20% by weight of the aerosol generating material. In some embodiments, the total amount of the aerosol former is from about 13% to about 16% by weight of the aerosol generating material. In some embodiments, the total amount of the aerosol former material is about 15% by weight of the aerosol generating material. When the aerosol generating material comprises an aerosol former in more than about 20%, by weight of the aerosol generating material, the aerosol generating may become “sticky” during storage, which may lead to processability issues.

In this context, an “aerosol former” is an agent, or material, that promotes the generation of an aerosol. An aerosol former may promote the generation of an aerosol by promoting an initial vaporisation and/or the condensation of a gas to an inhalable solid and/or liquid aerosol. In some embodiments, an aerosol former may improve the delivery of flavour from the aerosol generating material.

The aerosol former material has been found to improve the sensory performance of an article for use with an aerosol generation device comprising the aerosol generating material, by helping to transfer compounds such as flavour compounds from the fibrous material to the consumer. In some embodiments, the aerosol former material described herein is flavoured and/or comprises a flavour as described herein.

In general, any suitable aerosol former may be included in the aerosol generating material of the invention. Suitable aerosol formers include, but are not limited to: a polyol such as sorbitol, glycerol, and glycols like propylene glycol or triethylene glycol; a non-polyol such as monohydric alcohols, high boiling point hydrocarbons, acids such as lactic acid, glycerol derivatives, esters such as diacetin, triacetin, triethylene glycol diacetate, triethyl citrate or myristates including ethyl myristate and isopropyl myristate and aliphatic carboxylic acid esters such as methyl stearate, dimethyl dodecanedioate and dimethyl tetradecanedioate.

In some embodiments, the aerosol former is selected from the group consisting of glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, propylene carbonate, and mixtures thereof.

In some embodiments, the aerosol former comprises glycerol in amount from about 10% to about 90% by weight of the aerosol former.

In some embodiments, a ratio of aerosol former to fibrous material is from about 1:7 to about 1:3. For example, from about 1:6 to about 1:4, such as about 1:5. When the ratio of aerosol former to fibrous material is within this range, the aerosol generating material exhibits favourable storage properties and generates a favourable aerosol.

The aerosol-generating materials described herein may comprise a filler component. The filler component is generally a non-tobacco component, that is, a component that does not include ingredients originating from tobacco. The filler component may be a non-tobacco fibre such as wood fibre or pulp or wheat fibre. The filler component may also be an inorganic material such as chalk, perlite, vermiculite, diatomaceous earth, colloidal silica, magnesium oxide, magnesium sulphate, magnesium carbonate. The filler component may also be a non-tobacco cast material or a non-tobacco extruded material.

The filler component may be present in an amount from about 0 to about 20% by weight of the tobacco material, or in an amount of from about 1 to about 10% by weight of the aerosol-generating material. In a preferred embodiment, the aerosol-generating material comprises the filler component in an amount between about 5% and about 10% by weight of the aerosol-generating material. In some embodiments, the filler component is absent.

In some embodiments, the aerosol generating material has a thickness from about 250 μm to about 450 μm. In some embodiments, the aerosol generating material has a thickness from about 250 μm and about 400 μm, such as from about 280 μm to about 490 μm. In some embodiments, the aerosol generating material has a thickness of about 300 μm to about 400 μm. For example, the thickness may be from about 310 μm to about 390 μm. For example, the thickness may be from about 310 μm to about 380 μm. For example, the thickness may be from about 320 μm to about 370 μm. For example, the thickness may be from about 320 μm to about 360 μm. For example, the thickness may be from about 320 μm to about 350 μm. For example, the thickness may be from about 320 μm to about 340 μm. For example, the thickness may be from about 325 μm to about 340 μm. For example, the thickness may be from about 330 μm to about 340 μm.

In some embodiments, the aerosol generating material has a water content of between about 3% to about 8%. For example, the aerosol generating material has a water content of between about 4% to about 7%, for example between about 5% and about 6%. In some embodiments, the aerosol generating material comprises a water content of about 5%.

In some embodiments, the aerosol generating material has a filling value from about 2 cm³/g to about 10 cm³/g. In some embodiments, the aerosol generating material has a filling value of from about 3 cm³/g to about 8 cm³/g, for example the aerosol generating material may have a filling value of from about 4 cm³/g to about 7 cm³/g, such as from about 4 cm³/g to about 6 cm³/g. In some embodiments, the aerosol generating material has a filling value of about 5 cm³/g. When the filling value is between about 2 cm³/g to about 10 cm³/g, for example about 5 cm³/g, a consumable containing the aerosol generating material can weigh less. Therefore, the overall weight of material used, to achieve the same/required filling value, is less, which can provide a saving to the cost of goods. In addition, using the aerosol generating material described herein may also be advantageous because the resistance to draw is not affected, therefore providing the consumer with a favourable user experience.

In some embodiments, the aerosol generating material exhibits a bursting strength of between about 5 KPa and about 40 KPa. In some embodiments, the aerosol generating material exhibits a bursting strength of between about 10 KPa and about 30 KPa. In some embodiments, the aerosol generating material exhibits a bursting strength of between about 15 KPa and about 25 KPa, for example between about 20 KPa and about 25 KPa. In some embodiments, the bursting strength is about 20 KPa.

In an embodiment, the aerosol generating material is in the form of a rod. The aerosol generating rod may have a total weight of between about 250 mg and about 350 mg. In an embodiment, the aerosol generating rod may be wrapped in a wrapper having a permeability of less than 100 Coresta Units. The aerosol generating rod may have an outer circumference of at least about 19 mm, preferably between about 19 mm and about 23 mm or about 21 mm. This may facilitate insertion of the article into an aerosol generation device.

As used herein, the term “rod” is used to describe a generally cylindrical element of substantially circular, oval, or elliptical cross section.

In the compositions described herein, where amounts are given in % by weight, for the avoidance of doubt this refers to a dry weight basis, unless specifically indicated to the contrary. Thus, any water that may be present in the aerosol-generating material, or in any component thereof, is entirely disregarded for the purposes of the determination of the weight %. The water content of the aerosol-generating material described herein may vary and may be, for example, from 5 to 15% by weight. The water content of the aerosol-generating material described herein may vary according to, for example, the temperature, pressure and humidity conditions at which the compositions are maintained. The water content can be determined by Karl-Fisher analysis, as known to those skilled in the art. On the other hand, for the avoidance of doubt, even when the aerosol-former material is a component that is in liquid phase, such as glycerol or propylene glycol, any component other than water is included in the weight of the aerosol-generating material.

In some embodiments, the system comprises an article as described herein and the aerosol generation device is arranged to receive at least a portion of the article comprising the aerosol-generating material and to heat the portion of the article comprising the aerosol-generating material and generate an aerosol from the aerosol-generating material.

In some embodiments, the aerosol generating material may be positioned in an aerosol generating section of an article. When in use, the aerosol generating section may exhibit a pressure drop of from about 15 to about 40 mm H₂O. In some embodiments, the aerosol generating section exhibits a pressure drop across the aerosol generating section of from about 15 to about 30 mm H₂O.

In some embodiments, the article comprises a mouthpiece, and a cylindrical rod of aerosol generating material connected, either directly or indirectly, to the mouthpiece. A mouthpiece wrapper, also described herein as a tipping paper, may be wrapped around the full length of the mouthpiece and over part of the rod of aerosol generating material and has an adhesive on its inner surface to connect the mouthpiece and rod.

In some embodiments, the tipping paper extends 5 mm over the rod of aerosol generating material but it can alternatively extend between 3 mm and 10 mm over the rod 3, or more preferably between 4 mm and 6 mm, to provide a secure attachment between the mouthpiece and rod. The tipping paper can have a basis weight which is higher than the basis weight of plug wraps used in the article for use with an aerosol generation device, for instance a basis weight of 40 gsm to 80 gsm, more preferably between 50 gsm and 70 gsm, and in the present example 58 gsm. These ranges of basis weights have been found to result in tipping papers having acceptable tensile strength while being flexible enough to wrap around the article and adhere to itself along a longitudinal lap seam on the paper. The outer circumference of the tipping paper, once wrapped around the mouthpiece, may be about 21 mm.

According to some embodiments, there is provided an article for use in an aerosol provision system, the article including an aerosol generating material or substrate, a mouthpiece downstream of the aerosol generating material and a wrapper, wherein the wrapper comprises a sensate material. A portion of the mouthpiece wrapper close to the downstream end of the mouthpiece comes into contact with the consumer's lips during use.

The mouthpiece wrapper may be arranged to wrap around and enclose the mouthpiece in a region between the upstream end and downstream end of the mouthpiece. The mouthpiece wrapper may be arranged such that, when the article is inserted into a heating device, a portion of the mouthpiece wrapper is heated to the same or a similar temperature as the aerosol generating material.

In some embodiments, the mouthpiece wrapper comprises a sensate material. The sensate material may comprise a flavourant, as herein described. In some embodiments, the flavourant may suitably be liquorice, rose oil, vanilla, lemon oil, orange oil, a mint-flavour, suitably menthol and/or a mint oil from any species of the genus Mentha such as peppermint oil and/or spearmint oil, or lavender, fennel or anise. In preferred embodiments, the sensate material comprises sugars and/or sugar substitutes (e.g., sucralose, acesulfame potassium, aspartame, saccharine, cyclamates, lactose, sucrose, glucose, fructose, sorbitol, or mannitol). Additionally or alternatively, the sensate material may comprise a material that delivers a cooling, heating or sour sensation to the consumer during use of the article.

In some embodiments, the sensate material may comprise one or more of pH regulators, stabilizers, and/or antioxidants. These materials may help to increase the shelf-life of the mouthpiece wrapper and thus the article.

The sensate material can be encapsulated in an encapsulating material. For instance, the sensate material can be provided in the form of microcapsules which are applied to the wrapper.

The mouthpiece wrapper may comprise an inwardly facing surface and an outwardly facing surface and the sensate material may be present on at least a portion of the inwardly facing surface and/or the outwardly facing surface of the wrapper. For example, the sensate material may be disposed on an outwardly facing surface of the mouthpiece wrapper in an area which comes into contact with the consumer's lips during use. By disposing the sensate material on the outwardly facing surface of the mouthpiece wrapper, the sensate material may be transferred to the consumer's lips during use. Transfer of the sensate material to the consumer's lips during use of the article may modify the organoleptic properties (e.g. taste) of the aerosol generated by the aerosol generating substrate. For example, the sensate material may impart flavour to the aerosol generated by the aerosol generating substrate. The sensate material may be at least partially soluble in water such that it may be transferred to the user via the consumer's saliva.

Advantageously, a relatively low amount of sensate material may be required in order to modify the sensory properties delivered to the consumer during use of the article, although this will, to some extent, depend on the properties of the senate material because the minimum amount that is needed to modify the sensory properties will vary between different sensate materials. This may have the advantage that the addition of the sensate material to the mouthpiece wrapper may not significantly increase the overall weight of the article.

In some embodiments, there is described an aerosol generating material which is obtained or obtainable by a method as described herein. For example, such as the methods described in the claims.

### Example

An aerosol generating material was prepared having the composition illustrated in Table 1.

TABLE 1

| | |
|---|---|
| Botanical fibre | 60% |
| Wood pulp/wood fibre | 12% |
| Glycerol | 15% |
| CBD | 3.0% |
| Extract | 10% |

The aerosol generating material was prepared by adding rooibos to an extraction solvent and separated into a fibrous portion and an extract. The fibrous portion was mixed with wood pulp and refined to produce a refined pulp and formed into a sheet. The sheet was impregnated with glycerol and the extract to form an impregnated sheet. The impregnated sheet was dried to form an aerosol generating material. CBD was subsequently sprayed onto the surface of the aerosol generating material. 100 g of aerosol generating material was added to 500 mL of purified water, stirred for 30 mins, and the measured pH was 6.0.

The various embodiments described herein are presented only to assist in understanding and teaching the claimed features. These embodiments are provided as a representative sample of embodiments only, and are not exhaustive and/or exclusive. It is to be understood that advantages, embodiments, examples, functions, features, structures, and/or other aspects described herein are not to be considered limitations on the scope of the invention as defined by the claims or limitations on equivalents to the claims, and that other embodiments may be utilised and modifications may be made without departing from the scope of the claimed invention. Various embodiments of the invention may suitably comprise, consist of, or consist essentially of, appropriate combinations of the disclosed elements, components, features, parts, steps, means, etc, other than those specifically described herein. In addition, this disclosure may include other inventions not presently claimed, but which may be claimed in future.

1. An aerosol generating material comprising a fibrous material and a non-nicotine active.

2. The aerosol generating material according to claim 1, wherein the fibrous material comprises a non-tobacco botanical material, and wherein the non-tobacco botanical material is selected from the group consisting of fennel, star anise, mint, and rooibos, and mixtures thereof.

3. (canceled)

4. (canceled)

5. The aerosol generating material according to claim 1, wherein the aerosol generating material further comprises an aerosol former, and wherein the aerosol former is selected from the group consisting of glycerol, propylene glycol, diethylene glycol, triethylene glycol, tetraethylene glycol, 1,3-butylene glycol, erythritol, meso-Erythritol, ethyl vanillate, ethyl laurate, a diethyl suberate, triethyl citrate, triacetin, a diacetin mixture, benzyl benzoate, benzyl phenyl acetate, tributyrin, lauryl acetate, lauric acid, myristic acid, propylene carbonate, and mixtures thereof.

6. (canceled)

7. The aerosol generating material according to claim 1, wherein the aerosol generating material comprises a total fibrous material content of between about 70% and about 95% by weight on a dry weight basis.

8. The aerosol generating material according to claim 1, wherein the fibrous material comprises a first fibrous material and a second fibrous material,
wherein the first fibrous material and the second fibrous material are different,
wherein the first fibrous material comprises a non-tobacco botanical material and the second fibrous material comprises wood fibre and/or wood pulp, and
wherein a ratio between the first fibrous material and the second fibrous material is from about 10:1 to about 2:1.

9. (canceled)

10. (canceled)

11. The aerosol generating material according to claim 8, wherein the non-tobacco botanical material is selected from the group consisting of fennel, star anise, mint, and rooibos, and mixtures thereof.

12. (canceled)

13. (canceled)

14. The aerosol generating material according to claim 1, wherein upon contact with purified water, the aerosol generating material gives a pH of at most about 6.5, or between about 5 and about 8, or at least about 6.5.

15. (canceled)

16. (canceled)

17. The aerosol generating material according to claim 1, further comprising an extract derived from a non-tobacco botanical material,
wherein the non-tobacco botanical material is selected from the group consisting of fennel, star anise, mint, and rooibos, and mixtures thereof.

18. (canceled)

19. (canceled)

20. The aerosol generating material according to claim 17, wherein the aerosol generating material comprises the extract in an amount from about 5% to about 15% by weight of the aerosol generating material on a dry weight basis, and/or
wherein the aerosol generating material has a thickness of about 200 μm to about 450 μm, and/or
wherein the aerosol generating material has a filling value of between about 2 cm³/g to about 10 cm³/g, and/or
wherein the aerosol generating material exhibits a bursting strength of between about 5 KPa and about 40 KPa.

21. (canceled)

22. The aerosol generating material according to claim 5, wherein the aerosol former is included in an amount from about 10% to about 20% by weight of the aerosol generating material on a dry weight basis.

23. The aerosol generating material according to claim 5, wherein a ratio of aerosol former to fibrous material is from about 1:7 to about 1:3.

24. (canceled)

25. (canceled)

26. The aerosol generating material according to claim 1, wherein the aerosol generating material is free of, or substantially free of, tobacco material.

27. The aerosol generating material according to claim 1, wherein the non-nicotine active is a cannabinoid selected from the group consisting of cannabigerol (CBG), cannabichromene (CBC), cannabidiol (CBD), tetrahydrocannabinol (THC), cannabinol (CBN), cannabinodiol (CBDL), cannabicyclol (CBL), cannabivarin (CBV), tetrahydrocannabivarin (THCV), cannabidivarin (CBDV), cannabichromevarin (CBCV), cannabigerovarin (CBGV), cannabigerol monomethylether (CBGM), cannabinerolic acid, cannabidiolic acid (CBDA), cannabinol propyl variant (CBNV), cannbitriol (CBO), tetrahydrocannabinolic acid (THCA), and tetrahydrocannabivarinic acid (THCV A), and mixtures thereof, or
wherein the non-nicotine active is selected from the group consisting of caffeine, taurine, L-theanine, vitamin B1, vitamin B2, vitamin B5, vitamin B6, vitamin B7, vitamin C, ginseng extract, chamomile extract, lemon balm extract, and mixtures thereof.

28. (canceled)

29. (canceled)

30. (canceled)

31. The aerosol generating material according to claim 1, further comprising a pH modifier,
wherein the pH modifier is an organic acid selected from the group consisting of lactic acid, levulinic acid, benzoic acid, citric acid, 2-methylbutyric acid, and 2-methylvaleric acid, and mixtures thereof, or
wherein the pH modifier is a base.

32. (canceled)

33. (canceled)

34. (canceled)

35. An aerosol generating rod comprising an aerosol generating material according to claim 1.

36. An article comprising the aerosol generating material according to claim 1, wherein the article is configured such that, in use, a pressure drop across the aerosol generating section is from about 15 to about 40 mm H₂O.

37. The article according to claim 36, comprising an aerosol generating section comprising the aerosol generating material and a wrapper circumscribing the aerosol generating material,
wherein at least about 70% of a volume of the aerosol generating section comprises the aerosol generating material.

38. (canceled)

39. (canceled)

40. (canceled)

41. The article according to claim 37, wherein the article is for use in a delivery system, wherein the delivery system is a non-combustible aerosol provision system, and wherein the non-combustible aerosol provision system comprises an aerosol generator for insertion into the aerosol generating section.

42. (canceled)

43. (canceled)

44. The article according to claim 41, wherein the article is configured such that, in use, when the aerosol generator is received by the aerosol generating section of the article, the aerosol generator is in direct contact with at least a portion of the aerosol generating material.

45. A delivery system comprising an aerosol generating material according to claim 1.
